(19) United States
(12) Patent Application Publication
(10) Pub. No.: US 2021/0338887 A1
(43) Pub. Date: Nov. 4, 2021

(54) SYSTEMS FOR TARGETED TISSUE BIOSEALING OR REPAIR

(71) Applicant: THE TRUSTEES OF THE UNIVERSITY OF PENNSYLVANIA, Philadelphia, PA (US)

(72) Inventors: Jay Milan Patel, Philadelphia, PA (US); Claudia Loebel, Philadelphia, PA (US); Anthony R. Martin, Philadelphia, PA (US); Robert Leon Mauck, Philadelphia, PA (US); Jason A. Burdick, Philadelphia, PA (US)

(73) Assignee: THE TRUSTEES OF THE UNIVERSITY OF PENNSYLVANIA, Philadelphia, PA (US)

(21) Appl. No.: 17/259,758

(22) PCT Filed: Jul. 12, 2019
(86) PCT No.: PCT/US2019/041623

§ 371 Date: Jan. 12, 2021

Related U.S. Application Data
(60) Provisional application No. 62/697,252, filed on Jul. 12, 2018.

(51) Int. Cl. A61L 27/26; A61K 38/06; A61L 27/54

(52) U.S. Cl. CPC A61L 27/26; A61K 38/06; A61L 2430/06; A61L 2300/412; A61L 27/54

(57) ABSTRACT

The present disclosure relates to modified polymers, compositions comprising the same, and methods for repairing damaged tissue in a subject, including cartilage tissue. The modified polymers, compositions comprising the same, and methods described comprise one or more tunable characteristics such that the modified polymers, compositions comprising the same, and methods form an integrated microenvironment comprising one or more of viable cells, a fibrous barrier, or a bioseal at a site of damaged tissue in a subject.

### CROSS-REFERENCE TO RELATED APPLICATIONS

The present application claims the benefit of priority to U.S. Provisional Application No. 62/697,252, filed on Jul. 12, 2018, the entire contents of which are hereby incorporated by reference.

### TECHNICAL FIELD

The present disclosure relates to the field of tissue engineering and to the field of engineering of articular cartilage and fibrocartilage.

### BACKGROUND

Cartilage injuries represent one of the most common intra-articular knee injuries. Current surgical treatments provide short-term symptomatic relief, but often progress to joint-wide degeneration. Intra-articular mesenchymal stem cell (MSC) injections have become an increasingly common treatment strategy, with some data suggesting improved outcomes. Jo, Chris Hyunchul, et al. “Intra-articular injection of mesenchymal stem cells for the treatment of osteoarthritis of the knee: a proof-of-concept clinical trial.” Stem cells 32.5 (2014): 1254-1266. However, that process does not precisely localize MSCs to the injury site (Kim, Yong Sang, et al. “Comparative matched-pair analysis of the injection versus implantation of mesenchymal stem cells for knee osteoarthritis.” The American journal of sports medicine 43.11 (2015): 2738-2746), limiting efficacy and consistency of outcomes. Moreover, the fate and function of cells is not regulated post-injection, further restricting their regenerative potential. Thus, there is a need for a tunable biomaterial designed to attract stem cells or progenitor cells, including, for example, MSCs to—and to retain them at—damaged cartilage surfaces in order to enhance cartilage repair.

Cartilage damage is among the most common ailments of individuals in the U.S., especially considering the aging population. Tissue damage, particularly articular cartilage damage, can occur in pathologies such as arthritis and osteoporosis, as well as due to bone and/or joint injuries. Many of these tissues, among others, are unable to heal entirely after injury. This can lead to mechanical (e.g., wear) and biochemical (e.g., lack of nutrient delivery) defects to the surrounding area and surrounding tissues, which can exacerbate symptoms. Previous techniques to address these issues have used modification of biomaterials to enhance integration to adjacent cartilage or delivery of therapeutics. For example, Elisseeff and colleagues have described a chondroitin-sulfate based adhesive for corneal repair or integration of scaffolds to native cartilage tissue. Strehin, Iossif, et al. “A versatile pH sensitive chondroitin—sulfate—PEG tissue adhesive and hydrogel.” Biomaterials 31.10 (2010): 2788-2797; Reyes, Johann M G, et al. “A modified chondroitin sulfate aldehyde adhesive for sealing corneal incisions.” Investigative ophthalmology & visual science 46.4 (2005): 1247-1250; Wang, Dong-An, et al. “Multifunctional chondroitin sulphate for cartilage tissue—biomaterial integration.” Nature materials 6.5 (2007): 385; Yang, Shuqing, et al. “Use of a chondroitin sulfate bioadhesive to enhance integration of bioglass particles for repairing critical-size bone defects.” Journal of Biomedical Materials Research Part A 103.1 (2015): 235-242; U.S. Pat. No. 7,862,831 B2. However, these approaches were applied as adhesives directly to the site of injury. Moreover, the biomaterial adhesive in these previous studies was not used to generate a custom adhesive and mechanical microenvironment for cell attachment and differentiation, or with the goal of creating a cell-based biosealant. Furthermore, these biomaterial chemistries have not been utilized for the enhancement of targeted cellular delivery to defected tissue. Lastly, other biosealants have been developed (U.S. Pat. No. 6,183,498 B1), but not with a targeting mechanism, nor with enhanced delivery or recruitment of cells.

Consequently, there is a need for systems for targeted cartilage biosealing and/or repair which allow for cartilage healing and formation of a biomechanical seal to protect against further degeneration of surrounding tissue. In addition, there is need for convenient and effect methods of treatment and compositions that allow for targeted repair of cartilage tissue via local and/or systemic routes of administration.

### SUMMARY

The present disclosure is directed to methods of repairing damaged tissue in a subject, the methods comprising: administering to the subject a composition comprising a polymer, the composition further comprising one or more tunable characteristics such that the composition forms an integrated microenvironment having a desired biomechanical constitution at a site of damaged tissue in the subject. The one or more tunable characteristics can include, for example: tunable polymer composition; tunable polymer concentration; tunable polymer modification; tunable polymer conjugation; tunable polymer cross-linking; and/or tunable polymer stiffness.

In some embodiments, the methods disclosed herein further comprise ascertaining one or more aspects of the desired biomechanical constitution of the integrated microenvironment, and tuning one or more of the tunable characteristics of the composition comprising the polymer to give rise to the desired biomechanical constitution.

Also described herein are compositions for forming an integrated microenvironment at a site of damaged tissue in a subject, the compositions comprising a polymer, and wherein the compositions further comprise one or more tunable characteristics such that an integrated microenvironment having a desired biomechanical constitution is formed at the site of damaged tissue in the subject.

Also provided are biomaterials comprising a polymer that comprises one or more tunable characteristics such that an integrated microenvironment having a desired biomechanical constitution is formed at a site of damaged tissue in a subject

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In the drawings:

FIG. 1 depicts an exemplary experiment showing that mesenchymal stem cells (MSCs) respond differentially to tissue microenvironments, including degradation/digestion of extracellular matrix. Briefly, cartilage plugs were excised from bovine juvenile femoral condyle and cut to 100 μm transitional zone discs. Collagenase treatment (0.01%) was applied to transitional cartilage discs to mimic tissue degeneration. AFM (2.5 μm radius spherical tip) confirms softening of ECM mechanics. 500 MSCs were seeded onto discs for 24 hours. Cells were stained with phalloidin to visualize F-actin. Cell area and circularity (spreading and degree of adherence) were assessed by light microscopy. Noticeable change in cell spreading was observed with digestion. Digestion resulted in smaller, rounder cells: cell area decreased and circularity increased (p<0.05) with digestion.

FIG. 2 provides an exemplary schematic of a possible mechanism by which the biomaterial (i.e., the modified polymers and compositions described herein) of the disclosure conjugate to or bind to damaged tissue, including defected cartilage, allowing cell attachment and biosealant formation.

FIG. 3 provides an example image of a globular YAP/TAZ signal in a MSC interacting with a cellular microenvironment. Green represents the polymer matrix of the cellular microenvironment; magenta represents F-actin; and red represents transcriptional cofactors YAP/TAZ. It will be appreciated that the cellular morphology observed is that of a viable cell capable of differentiating and forming, for example, cartilage tissue.

FIG. 4 provides an example of the modified polymers and compositions of the disclosure targeting to a site of damaged tissue. An HA-Aldehyde-conjugated polymer is localized to an articular cartilage defect. The polymer is further conjugated with FITC-peptide for visualization via fluorescence.

FIG. 5 provides an exemplary schematic showing a transition from healthy cartilage tissue to progressive tissue degeneration. Top Row: Cartilage defects allow fluid flow and proteoglycan (PG) loss, causing adjacent tissue to experience elevated strain levels, leaving the tissue more susceptible to wear. Bottom Row: A biosealant system can restore fluid pressurization and has the potential to prevent, for example, osteoarthritis progression.

FIGS. 6A-6C provide the results of an assessment of the degree of infiltration of exemplary compositions according to the present disclosure into defected tissue.

FIGS. 7A-7D depict the results of an assessment of the quality of cellular adhesion to damaged tissue that was treated with inventive biomaterial conjugated with a chemoattractant molecule.

FIGS. 8A-8D depict the results of a further assessment of the quality of cellular adhesion to damaged tissue that was treated with inventive biomaterial, and demonstrate that cross-linking time can be tuned to elicit different cellular adhesion responses.

FIGS. 9A-9D depict the results of an assessment of the effect of the presence of inventive biomaterial on mechano-sensation when the biomaterial is conjugated with chemoattractant molecule.

FIGS. 10A-10D depict the results of a further assessment of the effect of the presence of inventive biomaterial on mechano-sensation, and demonstrate that cross-linking time can be tuned to elicit different mechano-sensation responses.

FIGS. 11A-11C provide the results of an assessment of the ability of the biomaterial to promote alpha smooth muscle (ASMA) fiber formation.

FIGS. 12A and 12B depict the results of an evaluation of the ability of cells to form a barrier at a damaged tissue interface to which inventive biomaterial was applied.

FIGS. 13A and 13B depict the results of an evaluation of the ability of inventive biomaterial to be retained at the site of damaged tissue in vivo.

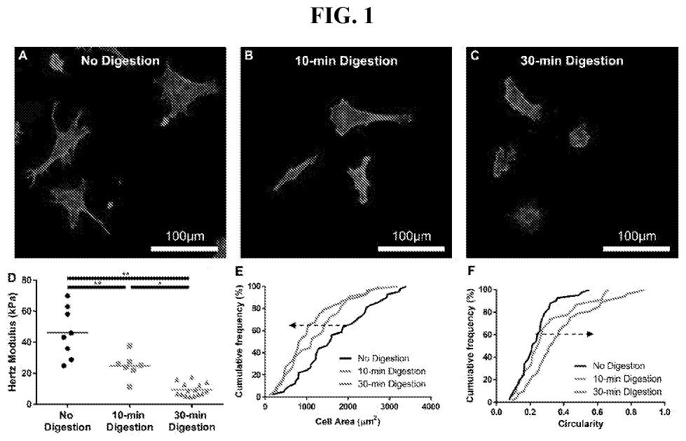

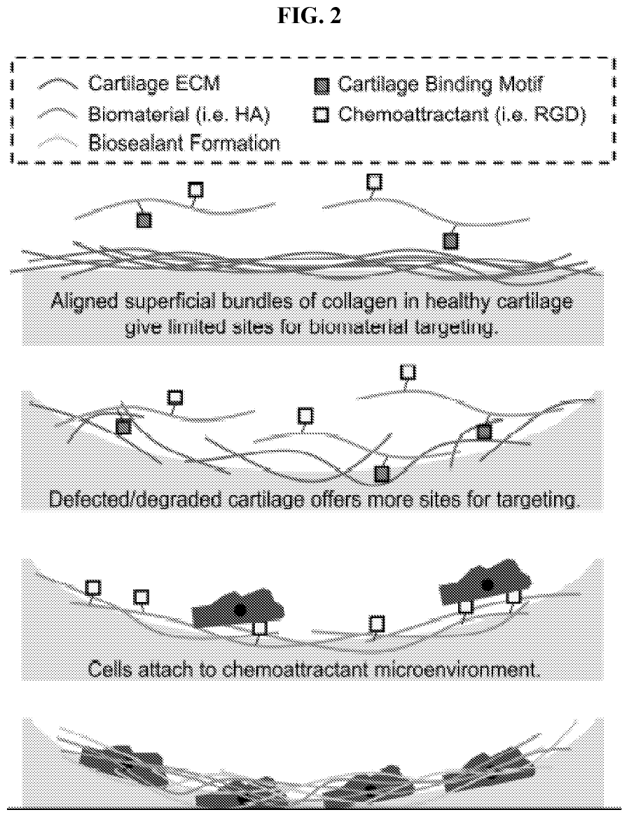

### DETAILED DESCRIPTION OF ILLUSTRATIVE EMBODIMENTS

The present subject matter may be understood more readily by reference to the following detailed description which forms a part of this disclosure. It is to be understood that this invention is not limited to the specific methods, conditions, or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treating”, “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder. This condition, disease or disorder can be cancer.

As employed above and throughout the disclosure the term “effective amount” refers to an amount effective, at dosages, and for periods of time necessary, to achieve the desired result with respect to the treatment of the relevant disorder, condition, or side effect. It will be appreciated that the effective amount of components of the present invention will vary from patient to patient not only with the particular compound, component or composition selected, the route of administration, and the ability of the components to elicit a desired result in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

As employed above and throughout the disclosure the term “sub-therapeutic amount” refers to an amount that is ineffective when administered as the sole therapeutic agent.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The term “administering” in reference to a “drug” or “therapeutic” or “therapeutic conjugate” means either directly administering a compound or composition as described herein, or administering a prodrug, derivative or analog which will form an equivalent amount of the active compound or substance within the body.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

The present disclosure is directed to modified polymers, compositions, and methods for use in tissue repair, such as articular cartilage repair. Methods of targeting the modified polymers to damaged tissue and creating a cellular microenvironment that integrates into the damaged or defected tissue and progresses cell behavior upon arrival are also disclosed.

Provided herein are compositions comprising modified polymers that can be applied to damaged or defected tissue in order to provide biosealing, promote repair, or both. The beneficial effects provided by the disclosed compositions can result, for example, from their ability to successfully provide one or more of: adhesion to the site of damage, diffusion into a damaged or defected area, prevention damage propagation, formation of a barrier at the interface between the tissue and the surrounding environment, formation of an integrated or interdigitating microenvironment at the site of damage, restoration of fluid pressurization at the site of damage, improvement of cellular adhesion, facilitation of cellular recruitment and attachment at the site of damage, promotion or modulation of cellular mechano-sensation at the site of damage, promotion of fibrotic cells at the site of damage, promotion of fibrogenesis at the site of damage, or promotion of matrix production at the site of damage.

The damage or defected tissue to which the present compositions can be applied can include cartilage, meniscus tissue, annulus fibrosis, ligament, tendon, or other tissue types.

The present polymers can be modified with one or more chemical groups. Such modifications can, for example, allow the polymers to be targeted to and covalently linked to defected tissue, including, for example, damaged cartilage tissue.

Additional modifications to the polymers may include therapeutic conjugates that bind to or otherwise interact with cells in the target location of damaged tissue. These therapeutic conjugates can include peptides and/or motifs of extracellular matrix proteins that mimic the biological activity of the extracellular matrix; chemoattractants; growth factors; anti-inflammatory reagents; antibiotics; therapeutics; and/or cell types (for example, stem cells or mesenchymal stem cells); these therapeutic conjugates can allow for greater cell recruitment, growth of desired cells at the sight of injury, and/or simply improve the endogenous repairs already present. Additionally, upon attachment, the polymers help produce a biosealant at the site of damaged tissue. The biosealant can be formed by a tunable microenvironment that can be optimized for tissue repair.

Thus, described herein are modified polymers to target damaged tissue and create a microenvironment that programs cell behavior upon arrival. A composition comprising a polymer modified with a chemical group (e.g., aldehyde, tyramine, methacrylate, and others disclosed herein) is disclosed.

Further disclosed herein are methods of using controlled biochemical synthesis techniques for generating the modified polymers. Examples of synthesis techniques that can be used for generating the modified polymers described herein include, but are not limited to sodium periodate oxidation, methacrylic anhydride methacrylation, peptide conjugation via Michael addition, Norbornene modification (BOP coupling), Carbodiimide coupling (e.g., tyramine), and others that will be appreciated by persons of skill in the art of polymer synthesis of modification.

In some embodiments, the modified polymers are conjugated with a chemoattractant molecule/peptide (e.g., an RGD peptide) and/or drug. Thus, one modification allows for covalent linkage to defected tissue, and the other allows for recruitment of cells, promotes a cue for cell response, or both.

The modified polymers, compositions, and methods described herein are highly tunable (with regards to stiffness and other parameters described herein), which can affect cell behavior (for example, adhesion, spreading, and differentiation). The modified polymers, compositions, and methods of the disclosure promote attachment of cells at a targeted site of tissue damage and promote fibrous tissue formation, creating a tunable biosealant system at the site of tissue damage.

The mechanical properties of the modified polymers can also be tuned to promote new tissue formation of the desired lineage (e.g., chondrogenesis, osteogenesis, and others that will be appreciated by a person skilled in the art). The biomaterial can also be conjugated with chemical cues (e.g., anti-inflammatory or growth factors) to enhance and guide repair.

In some embodiments, the modified polymers, compositions, and methods can be applied to focal or debrided articular cartilage defects; an injectable or arthroscopically-applicable biosealant system can thereby restore fluid pressurization and reduce proteoglycan loss from damaged surfaces following cartilage injury. The modified polymers, compositions, and methods of the disclosure can also be used for targeted tissue repair through cell response to biomaterial stiffness and/or drug release. In addition, the modified polymers, compositions, and methods have the capacity to be used in other applications including meniscus, ligament, tendon, intervertebral disc, and other tissues that will be appreciated by persons of skill in the art, to form targeted fibrous barriers or enhance repair in other defected tissues.

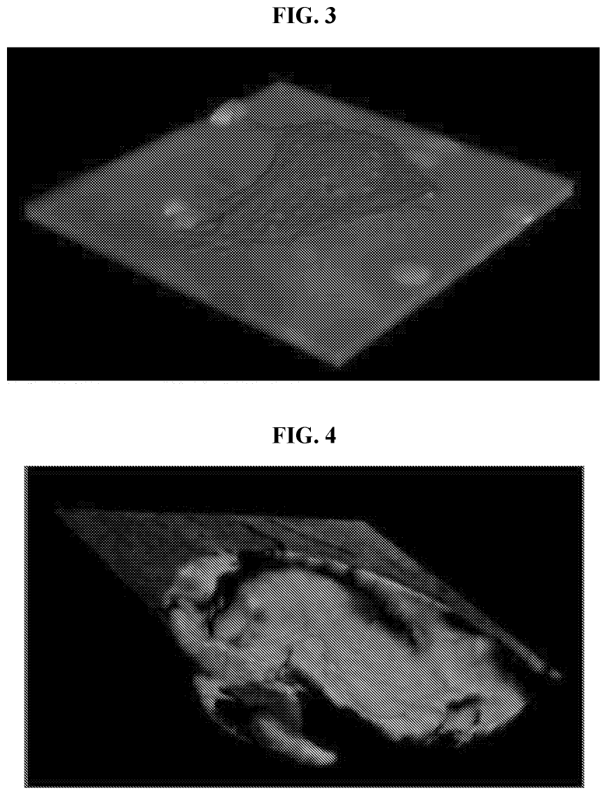

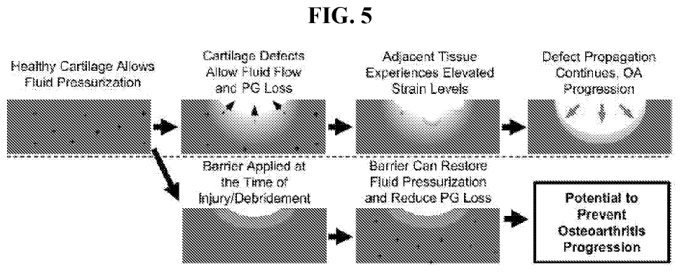

The present disclosure provides a therapeutic to address progressive tissue degeneration following focal injury. Many tissues of the body are unable to heal after defects form, and tissue adjacent to these defects is often susceptible to mechanical and biochemical changes as a consequence of the interrupted tissue structure. For example, focal cartilage defects compromise the fluid pressurization of adjacent tissue by introducing new boundaries that are open to fluid flow, leading to increased strain levels and physiochemical flow of proteoglycans, significantly altering the biochemistry of the tissue. These degenerative changes in adjacent tissue leave it extremely vulnerable to wear, initiating a vicious cycle that increases defect size and that ultimately concludes with joint-wide osteoarthritis (OA) (see FIG. 5). Similar degenerative changes are seen in other tissues of the body, such as ligament, tendon, and meniscus, where tears and other tissue interruptions progress to frank failure of the tissue.

The present disclosure relates to 1) targeted delivery of a cellular microenvironment to damaged tissue, and 2) using the cell-attracting and tunable cellular microenvironment to promote the formation of a biosealant and/or to elicit a desired tissue response.

In some embodiments, the targeting mechanism for targeting modified polymers to a site of damaged tissue in a subject is used to form a biosealant at the site of damaged tissue. In other embodiments, it is used to enhance delivery of therapeutics/cells to the site of defected tissue, and in general, enhance repair. Rather than conjugating the polymer with chemoattractant molecules, it can be conjugated with growth factors or drugs to elicit a desired cellular response. Moreover, the adhered coating can be conjugated with cross-linkable chemical groups (e.g., methacrylate), tuning the microenvironment to the desired neo-tissue formation.

Also disclosed herein are methods of treating or repairing damaged are defected tissue in a subject comprising administering the modified polymers and/or compositions described herein to the subject. In some embodiments, the modified polymers and compositions are administered by injection following injury. In the case of articular cartilage injuries, the methods of treating can be applied arthroscopically near the defect, or even via systemic injection. The methods of treating can also be used in conjunction with stem cell injections to improve delivery, repair, and/or regeneration of defected or damaged tissue in the subject. These approaches can be extended to a wide array of musculoskeletal tissues (meniscus, ligament, tendon, disc), as well as other tissues (dura, pericardium, abdominal wall) throughout the body. Ultimately these therapies enhance existing treatments that either remove damaged tissue (i.e. cartilage debridement, meniscectomy) or repair tissue (i.e. intra-articular MSC injection, meniscus repair, tendon repair).

The described modified polymers, compositions, and methods enable targeting of the polymer to specific tissues via cartilage binding motifs; they further enable tuning of the polymers, compositions, and methods with, for example, specific cell attracting agents to improve cartilage repair; they further enable fibrogenesis and fibrous bioseal formation at a site of damaged tissue in a subject, providing an integrated microenvironment conducive to its healing.

### EXAMPLES

### Example 1

Methods: Hyaluronic acid (HA; 65 kDa) was methacrylated (˜70% modification) and conjugated with fluorescent (FITC) and cell-adhesive (RGD) groups. The material was oxidized with sodium periodate to modify adjacent hydroxyl groups into aldehydes (˜30% substitution), chemical groups that can form covalent linkages with exposed amines in damaged tissue. Wang, Dong-An, et al. “Multifunctional chondroitin sulphate for cartilage tissue—biomaterial integration.” Nature materials 6.5 (2007): 385. To investigate material attachment to native tissue, the superficial zone was removed from 6 mm-diameter cylindrical cartilage plugs to expose “damaged” cartilage. These plugs were then sectioned into 100 μm thick discs. The material was applied, discs were rinsed in PBS, and fluorescence was quantified (n=4 per group). Material attachment was monitored as a function of clinically-relevant application times (5, 10, 30 minutes). To assess any potential cytotoxicity to chondrocytes in the host tissue, viability was also evaluated after treatment. Cartilage plugs were incubated in biomaterial solution for 30 minutes, followed by culture for 24 hours, at which point Live/Dead staining was performed. To determine whether the material supported cell adhesion, Cell Tracker labeled bovine juvenile MSCs were seeded onto material-coated constructs for 24 hours and imaged.

Results: In initial studies, the material showed preferential attachment to the defected area. Biomaterial attachment increased with duration of application, with 30-minute application showing ˜50% greater fluorescence intensity than 10-minute application. Modification for 30 minutes did not alter cell viability (91+3%) compared to PBS controls. Labeled cells adhered to and began spreading on the biomaterial coating within 24 hours.

Conclusion: These results show that a novel microenvironment can be adhered to cartilage defects. Moreover, the material was cytocompatible and cell adhesive. Future work will further optimize biomaterial attachment, cell adhesion and spreading, and microenvironment mechanics to promote chondrogenic differentiation of adherent stem cells.

### Example 2—Infiltration into Damaged Tissue

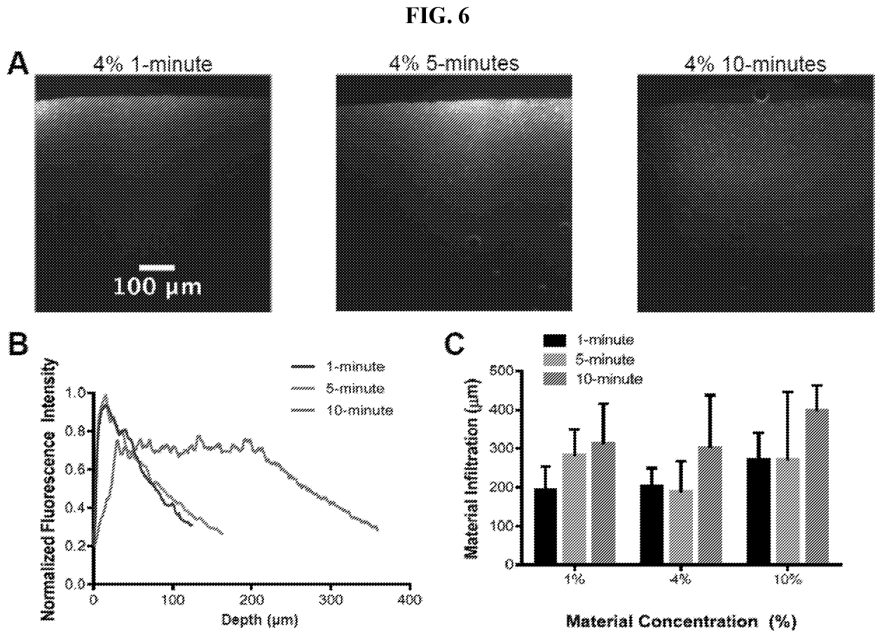

Compositions comprising various concentrations (1%, 4%, and 10%) of hyaluronic acid biomaterial that had been modified as disclosed in Example 1 (i.e., to include aldehyde groups and methacrylate groups) were applied onto cartilage plugs for varying times (1, 5 and 10 minutes), and the degree of infiltration of the biomaterial into the underlying cartilage was assessed. The degree of infiltration was calculated by determining the maximum intensity of fluorescence, detecting the surface of the damaged tissue at 20% of this maximum intensity, and marking the depth of infiltration as the point at which the fluorescent intensity falls below 20%. As shown in FIG. 6A-C, the depth of infiltration increased with concentration and application time. FIG. 6A shows fluorescence images and graphical depictions of infiltration by biomaterial (4% concentration) after 1, 5, and 10 minutes. FIG. 6B represents a graph showing the normalized fluorescence activity as a function of depth of infiltration over time. FIG. 6C is a graph showing the depth of infiltration over time for 1%, 4%, and 10% biomaterial samples. Infiltration of the biomaterial into the damaged tissue can be referred to as an integrated microenvironment, and can function to resist the shear forces of a joint, thereby preventing delamination to which a simple surface coating can be vulnerable. Thus, the present compositions can produce an integrated or interdigitating microenvironment at the site of tissue damage.

### Example 3—Improvement of Cellular Adhesion

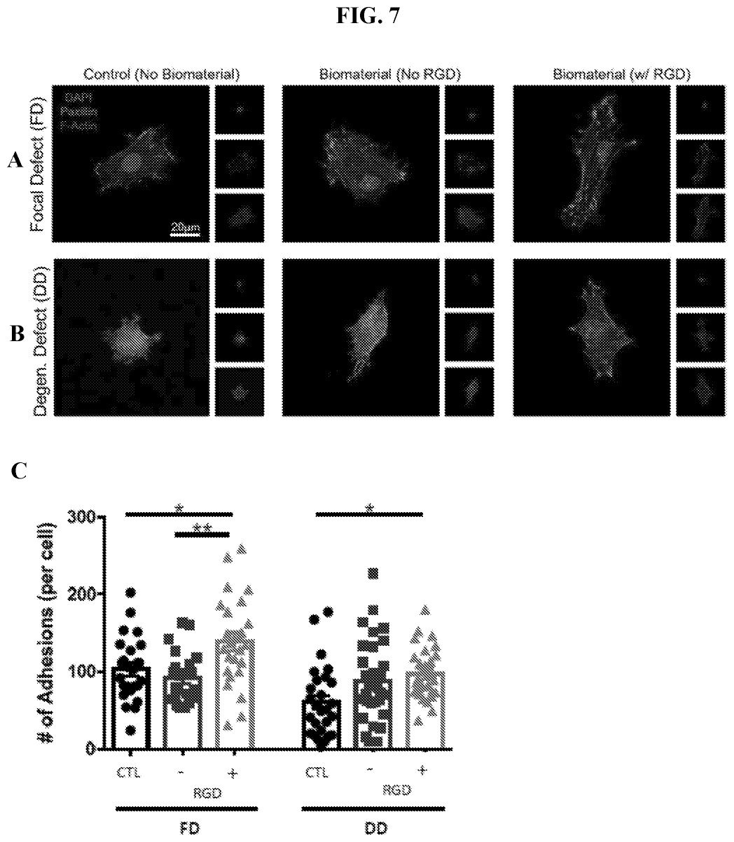

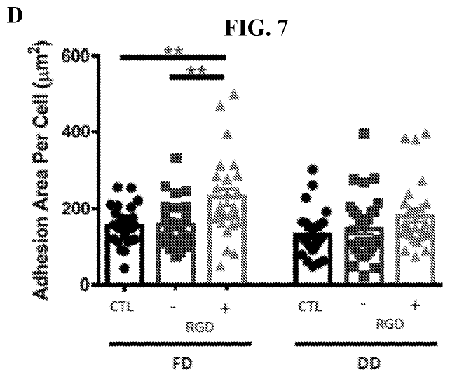

Biomaterial as described in Example 1 (i.e., containing aldehyde groups and methacrylate groups, and conjugated with RGD groups) and a second sample that was the same except not including RGD groups were separately applied to non-digested (ND, also referred to as a focal defect, FD) cartilage and digested (D—also referred to as a degenerated defect, DD) cartilage, respectively. The quality of cellular adhesion to the tissue surface was then assessed using paxillin and F-actin staining. FIG. 7A shows the results of staining with respect to the ND sample, FIG. 7B shows the results of staining with respect to the D sample, FIG. 7C is a graph showing how the number of adhesions per cell varies among samples, and FIG. 7D is a graph showing how the adhesion area per cell varies among samples. The results revealed that RGD peptide presentation improves the quality of cellular adhesion to defected cartilage.

### Example 4—Improvement of Cellular Adhesion II

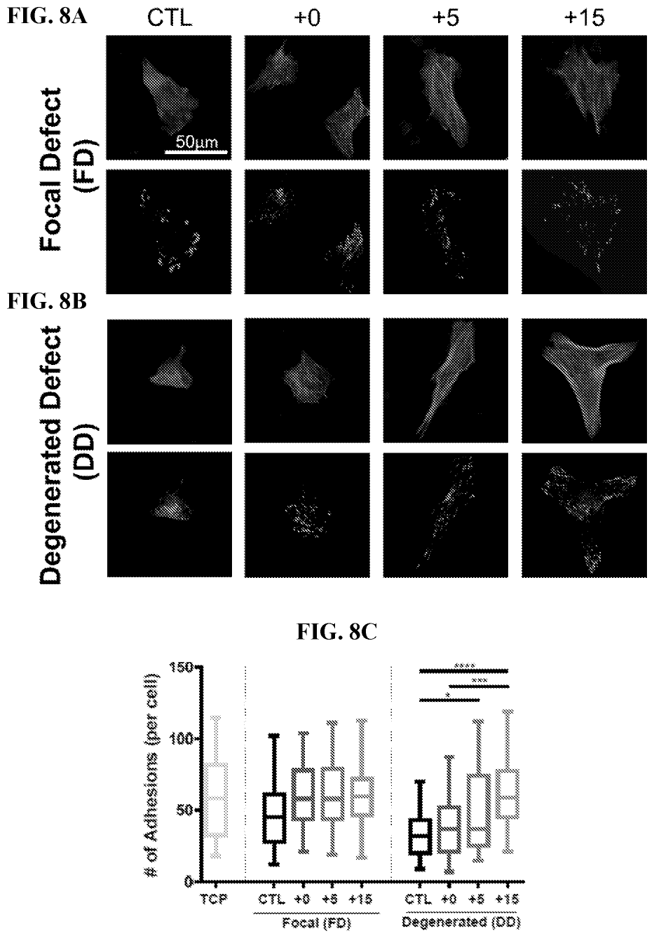

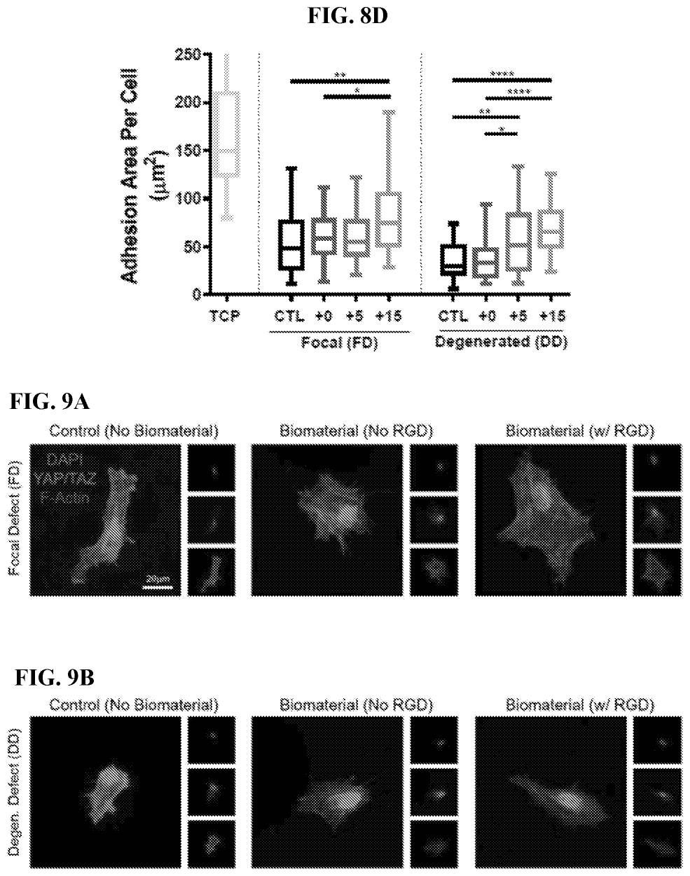

Cellular adhesion to tissue to which biomaterial as described in Example 1 was applied was assessed as a function of time of crosslinking of the biomaterial. Both focal defect (FD) and degenerated defect (DD) tissue was assessed, and the results are depicted in FIGS. 8A and 8B, respectively, for control samples (no biomaterial), as well as for biomaterial samples respectively subjected to 0, 5, and 15 minutes crosslinking. Red staining was for paxillin, and purple staining was for tissue culture plastic (TCP). FIGS. 8C and 8D show that number of adhesions and adhesion area per cell increase with crosslinking time, thereby demonstrating one aspect of the tunability of the present biomaterials.

### Example 5—Effect of Biomaterial on Mechano-Sensation

Biomaterial as described in Example 1 (i.e., containing aldehyde groups and methacrylate groups, and conjugated with RGD groups) and a second sample that was the same except not including RGD groups were separately applied to non-digested (FD, also referred to as a non-digested defect, ND) cartilage and degenerated defect (DD, also referred to as a digested defect, D) cartilage, respectively. The quality of cellular mechano-sensation with respect to the tissue surface was then assessed using staining for two nuclear cofactors (YAP and TAZ) and F-actin staining. The ratio of signal in the nucleus to signal in the cytoplasm can be was calculated in order to provide an indication of cellular mechano-sensation, which can be a driver of MSC behavior.

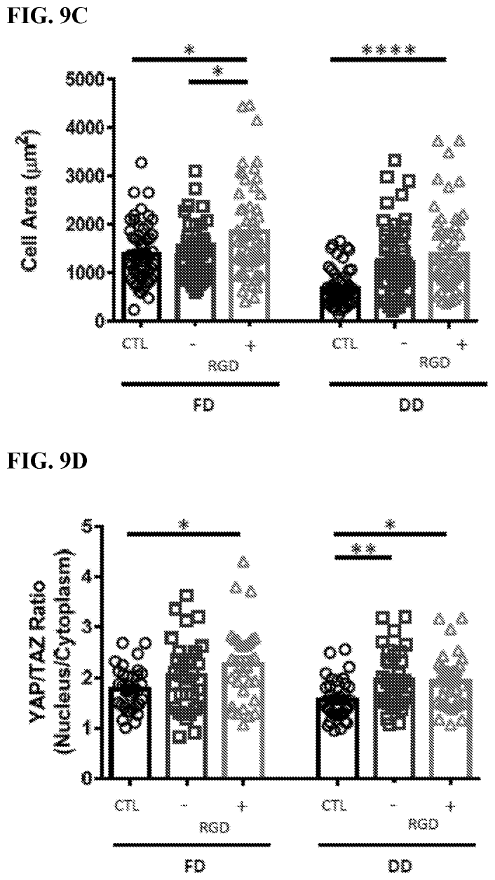

FIG. 9A shows the results of staining with respect to the FD sample, FIG. 9B shows the results of staining with respect to the DD sample, FIG. 9C is a graph showing how the cell area varies among samples, and FIG. 9D is a graph showing how YAP/TAZ ratio varies among samples. The results revealed that RGD peptide presentation improves the quality of cellular mechano-sensation. In particular, cell area and YAP/TAZ nuclear localization increase with biomaterial application and with RGD presentation.

### Example 6—Effect of Biomaterial on Mechano-Sensation II

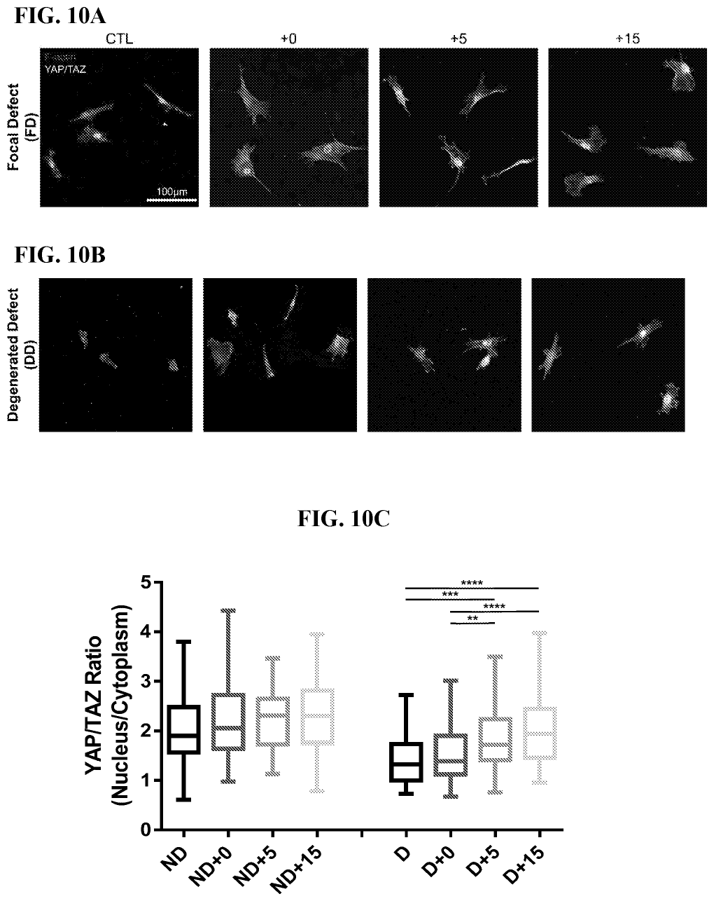

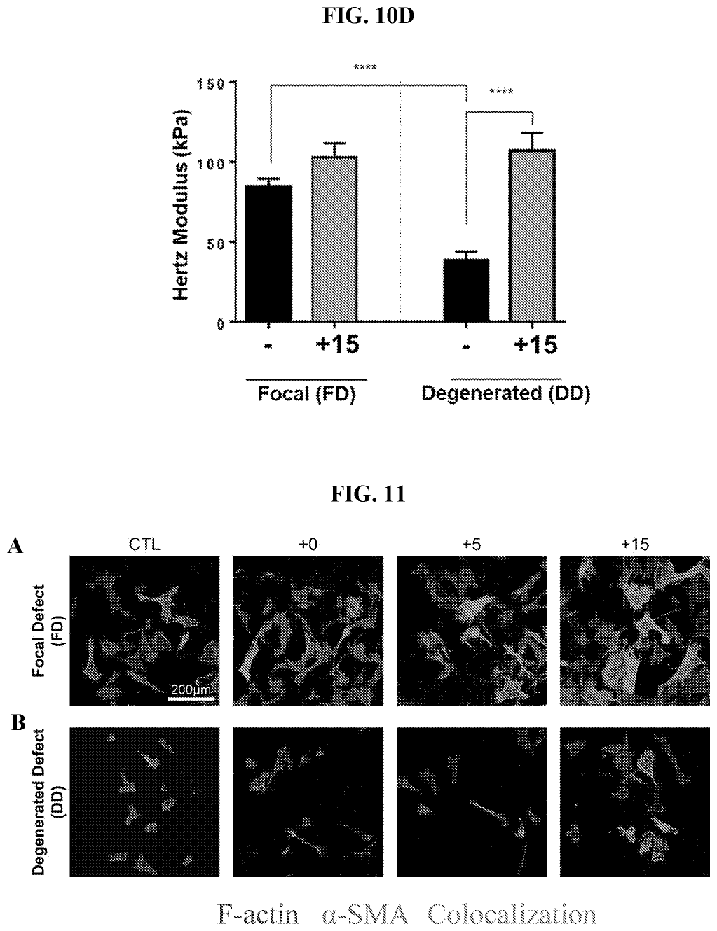

Mechano-sensation was assessed as a function of time of crosslinking of the biomaterial. Biomaterial as described in Example 1 was applied to both focal defect (FD) and degenerated defect (DD) tissue, and results are depicted in FIGS. 10A and 10B, respectively, for control samples (no biomaterial), as well as for biomaterial samples respectively subjected to 0, 5, and 15 minutes crosslinking. White staining was for YAP/TAZ, and purple staining was for F-actin. FIGS. 10C and 10D respectively show that YAP/TAZ nuclear localization (ratio between nucleus and cytoplasm) and hertz modulus (measure of cell area) increase with crosslinking time, thereby demonstrating another aspect of the tunability of the present biomaterials.

### Example 7—Effect on Promoting Fibrotic Cells or Fibrogenesis

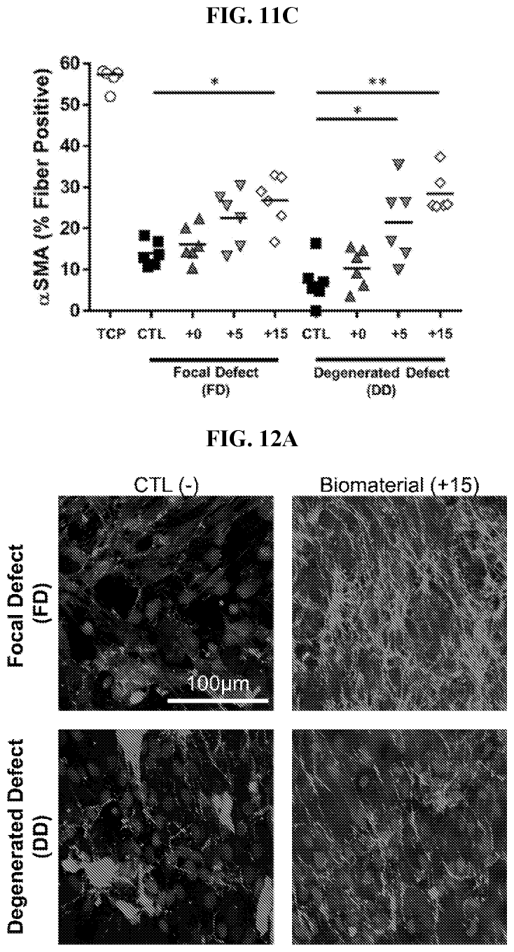

One indicator of a pro-fibrotic environment is alpha smooth muscle actin (ASMA or α-SMA) fiber formation. Following application of biomaterial as described in Example 1 to FD and DD tissue, ASMA fiber formation was assessed for co-localization with F-actin using fluorescent staining protocols (purple for F-actin, red for ASMA, with pink indicating co-localization), and the percentage of cells that were ASMA-positive were calculated. FIGS. 11A and 11B show the results of the assessment with respect to FD and DD tissue, respectively for control samples, as well as for biomaterial samples respectively subjected to 0, 5, and 15 minutes crosslinking. FIG. 11C provides data confirming that the percentage of ASMA-positive cells increases in both FD and DD tissues with biomaterial application and crosslinking time.

### Example 8—Improvement of Matrix Production

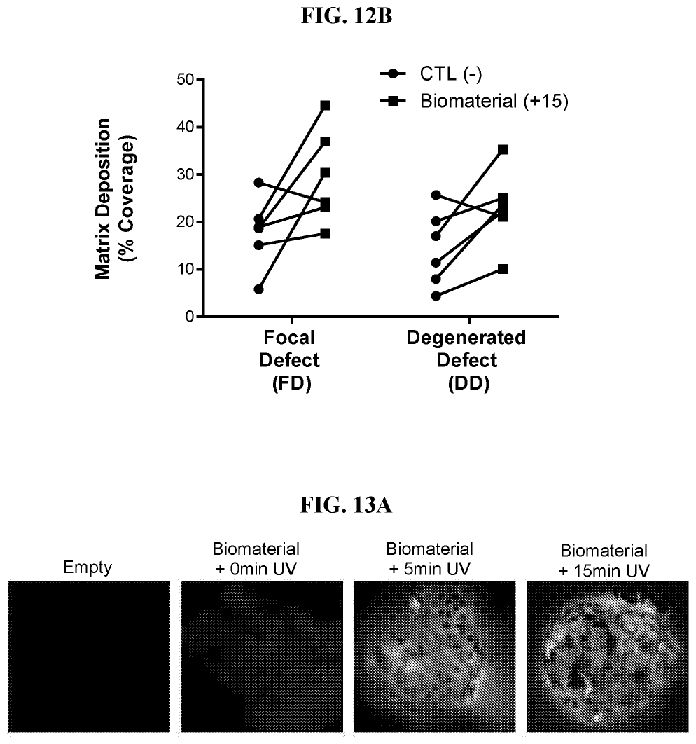

In order to evaluate the ability of cells to form a barrier at a damaged tissue interface to which inventive biomaterial was applied, a protocol was used to visualize newly-synthesized matrix. In cell culture medium, L-methionine was replaced with L-azidohomoalanine (AHA). Given the similar chemical structure of AHA to L-methonine, cells incorporate AHA into newly-synthesized proteins. An azide group on the AHA molecule allows for the cycloaddition (staining) of a dye-conjugated molecule, allowing for visualization of new matrix. FIG. 12A shows how application of biomaterial as described in Example 1 and subjected to 15 minutes of crosslinking increases ASMA-positive cells, and thereby matrix production, in both FD and DD tissues, as compared with control samples (to which no biomaterial was used). FIG. 12B provides data resulting from measurement of percentage of coverage on FD and DD tissue by ASMA-positive cells (thereby indicating matrix deposition), following application of no biomaterial (control) or biomaterial as described in Example 1 and subjected to 15 minutes of crosslinking.

### Example 9—Retention of Biomaterial at Damaged Tissue

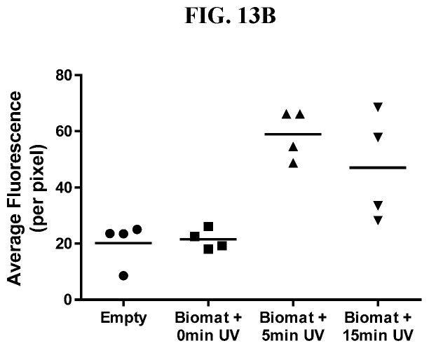

In order to assess the ability of biomaterial as described in Example 1 to be retained at the site of damaged tissue to which it is applied, four separate partial thickness defects were formed within trochlear (cartilaginous) tissue in the knee of respective juvenile Yucatan minipig subjects, and the four defects were either untreated (empty) or coated with biomaterial (one each of no crosslinking, 5 minutes UV crosslinking, or 15 minutes UV crosslinking). Seven days after treatment, in vivo assessments were made concerning the degree of retention of biomaterial at the defect site. FIG. 13A provides the results of fluorescence imaging of the four defects (control, biomaterial with no crosslinking, biomaterial with 5 min crosslinking, biomaterial with 15 minutes crosslinking), and FIG. 13B provides the results of measurements of fluorescence at the respective defects. These results indicate that biomaterial was retained after 7 days in vivo, especially with respect to the samples that had been subjected to crosslinking.

### EMBODIMENTS

Embodiment 1. A method of repairing damaged tissue in a subject, comprising: administering to the subject a composition comprising a polymer, the composition further comprising one or more tunable characteristics such that the composition forms an integrated microenvironment having a desired biomechanical constitution at a site of damaged tissue in the subject.

As used throughout the present disclosure, an “integrated microenvironment” may be a region within a tissue that is adjacent to a tissue/environment interface that is characterized by interdigitation, interdispersion, or mixture between endogenous tissue components and biomaterial (i.e., the present compositions, or the polymer of the compositions) in accordance with the present disclosure. FIG. 6A, for example, shows how biomaterial according to the present disclosure infiltrates into and disperses within endogenous tissue following application to a surface thereof, thereby forming an integrated microenvironment. The resulting microenvironment can represent a new surface environment at the site of application of the composition to the tissue that can promote cell attachment, influence mechno-sensation, and/or direct cell response (e.g., promote matrix deposition) by providing biomechanical and/or biochemical support to cells at, on, or in the microenvironment. FIG. 3, for example, shows how cells can attach to the surface of the tissue region at which the integrated microenvironment is located, and Examples 3-7, above, demonstrate that the present compositions that are used to form the integrated microenvironment can be tuned in order to influence cellular response at the integrated microenvironment.

The one or more tunable characteristics of the composition can be tuned by a person skilled in the art to enable an integrated microenvironment that favors cell recruitment, cell growth, and/or cellular differentiation according to the present disclosure. As used herein, “tunable” means capable of being adjusted to have certain desired characteristics or properties.

Embodiment 2. The method of Embodiment 1, wherein the one or more tunable characteristics comprise: a tunable polymer composition; a tunable polymer concentration; a tunable polymer modification; a tunable polymer conjugation; a tunable polymer cross-linking; or a tunable polymer stiffness. The tunable characteristics can be selected based, for example, on a particular target tissue, on the biomechanical properties of the target tissue, on the biochemical properties of the target tissue, or on the desired characteristics or properties of the integrated microenvironment to be formed at the site of damaged tissue.

Regarding the tunable polymer composition, the polymer, the polymer concentration, and the polymer molecular weight can be tuned to control cellular stiffness of the cellular microenvironment and cellular response. For example, the concentration of polymer in the composition can be tuned, allowing for the amount of polymer added to and the ultimate density and mechanics of the microenvironment to be controlled. Polymer concentration may be, for example, from 0.1-99% w/v (e.g., g/mL) within the composition. In certain embodiments, the polymer concentration is about 0.1-50%, 0.1-40%, 0.1-30%, 0.1-20%, 0.1-10%, or 0.1-5% w/v within the composition. For example, the polymer concentration may be about 0.1, 0.2, 0.3, 0.4, 0.5, 0.6, 0.7, 0.8, 0.9, 1, 2, 3, 4, 5, 6, 7, 8, 9, 10, 12, 14, 15, 16, 18, 20, 22, 24, 26, 28, 30, 32, 34, 36, 38, 40, 42, 44, 46, 48, 50, 55, 60, 65, 70, 75, 80, 85, 90, or 95% w/v within the composition. Additionally or alternatively, the molecular weight can also be tuned, altering diffusion of the polymer into the damaged tissue (e.g., the cartilage tissue), and ultimately the foundation of the integrated microenvironment.

Regarding the tunable polymer modification, the polymers can be modified with a peptide. The peptide concentration and type (RGD, HAVDI) can be tuned to control cellular response. For example, adding RGD groups to the polymer promotes cellular attachment and spreading. Other peptides containing proteins or growth factors can tune stem cells to differentiate toward a desired phenotype (e.g. chondrogenesis, fibrogenesis, osteogenesis, etc.). Any peptide with a thiol can be added to the polymers. Peptides that have a biological activity of an extracellular matrix protein are particularly desirable. An activity of a protein can be a biolophysical or biomechanical attribute of an endogenous protein or peptide in the cellular or extracellular environment. A peptide that has a biological activity of an extracellular matrix peptide is a peptide, motif, or residue that physically and/or biochemically acts like the extracellular matrix protein from which it is derived, thereby mimicking the protein. Extracellular matrix proteins with desirable biological activities include, for example: Fibronectin (RGD, GRGDSP, PHSRN), N-Cadherin (HAVDI), Heparin (Dextran sulfate), Collagen (IVH1, DGEA), Laminin (IKVAV, YIGSR).

Regarding the tunable stiffness: the stiffness of the cellular microenvironment can control the response of adhered stem cells through mechano-sensation. In addition, mechanics of degenerated tissue can be at least partially restored or fully restored by tuning the stiffness of the cellular microenvironment. Stiffness of the microenvironment is typically measured via atomic force microscopy (AFM), providing mechanics at the cell-scale level. A chemical modification can be selected based on desired properties of the cellular microenvironment. For example, methacrylation and/or norbornene modification can be used to tune the amount of cross-linking. Cross-linking can be induced using, for example, ultraviolet light or blue light, or cross-linking can be chemically induced. In addition, the duration of cross-linking time can be tuned to achieve a desired stiffness of the cellular microenvironment. For example, the material can be subjected to cross-linking for about 1 to about 30 minutes, such as for 1, 3, 5, 7, 9, 10, 12, 14, 15, 17, 18, 20, 22, 24, 25, 26, 28, or 30 minutes. Increased methacrylation of the polymers will cross-link to a higher stiffness than polymers having relatively less methacrylation. Based on degree of methacrylation and UV cross-linking time, the stiffness of the biomaterial itself can be varied from <1 to >50 kPa. When applied to either non-degraded or degraded tissue, the cross-linked biomaterial can provide 50-100% improvement in mechanics. The polymers can be modified to have aldehyde substitutions. The adjacent hydroxyl groups on the polymers can be modified to present aldehydes, which can bind to exposed amines in damaged tissues. The degree of aldehyde substitution (and/or tyramine in an alternative embodiment) can be varied to modify binding to the exposed damaged tissue. Tyramine substitution can also be conjugated to the backbone of the polymers, and allow for a range of attachment to damaged cartilage, for example, thereby cross-linking of the integrated microenvironment, and conjugation of peptides.

Embodiment 3. The method of Embodiment 1 or 2, further comprising ascertaining one or more aspects of the desired biomechanical constitution of the integrated microenvironment, and tuning one or more of the tunable characteristics of the composition comprising the polymer to give rise to the desired biomechanical constitution. Aspects of the desired biomechanical constitution can be, for example, stiffness, cellular make-up, cellular density, extracellular matrix composition, innervation, and/or ability to support angiogenesis. A person skilled in the art will readily appreciate and be able to ascertain one or more aspects of a desired biochemical constitution of an integrated microenvironment by, for example, understanding the tissue type and/or the type and extent of damage or injury to the tissue.

Embodiment 4. The method of any one of Embodiments 1-3, wherein the polymer comprises a biopolymer, a polysaccharide, or a synthetic polymer, or combinations thereof

Embodiment 5. The method of Embodiment 4, wherein the biopolymer comprises collagen, gelatin, albumin, cellulose, or any combination thereof

Embodiment 6. The method of Embodiment 4, wherein the polysaccharide comprises hyaluronic acid, dextran, chondroitin sulfate, heparin, dermatan, or any combination thereof

Embodiment 7. The method of Embodiment 4, wherein the synthetic polymer comprises polyethylene glycol (PEG), poly-L-lactic acid (PLLA), polylacticglycolic acid (PLGA), polyglycolic acid (PGA), polycaprolactone (PCL), polyethylene oxide (PEO), polyvinyl alcohol (PVA), polyethylene, polypropylene, a polyacrylate, a polycarbonate, or any combination thereof

Embodiment 8. The method of any one of Embodiments 2-7, wherein the tunable polymer modification comprises an aldehyde modification, a methacrylate modification, a norbornene modification, or a tyramine modification. Methods useful for producing polymers having the modifications described herein include, but are not limited to, sodium periodate oxidation, methacrylic anhydride methacrylation, peptide conjugation via Michael addition, norbornene modification (BOP coupling), and carbodiimide coupling (e.g tyramine).

Embodiment 9. The method of any one of Embodiments 2-8, wherein the tunable polymer cross-linking comprises a cross-linkable polymer, a cross-linkable polymer modification, or a cross-linking agent.

Embodiment 10. The method of any one of Embodiments 1-9, wherein the polymer comprises a therapeutic conjugate.

Embodiment 11. The method of Embodiment 10, wherein the therapeutic conjugate comprises a drug. The drug can be, for example, an anti-inflammatory, an anti-histamine, an anti-biotic, transforming growth factor (TGF)-β, (SDF), bone morphogenic protein (BMP), or a pain killer. Appropriate drugs will be readily appreciated by persons skilled in the art.

Embodiment 12. The method of Embodiment 10, wherein the therapeutic conjugate comprises a peptide having a thiol group.

Embodiment 13. The method of Embodiment 12, wherein the peptide comprises an RGD cell adhesion domain.

Embodiment 14. The method of Embodiment 12, wherein the peptide is a growth factor.

Embodiment 15. The method of Embodiment 12, wherein the peptide comprises an extracellular matrix peptide or a derivative of an extracellular matrix peptide.

Embodiment 16. The method of Embodiment 15, wherein the extracellular matrix peptide comprises one or more peptide activities of fibronectin, N-cadherin, heparin, collagen, or laminin, or combinations thereof. A peptide activity can be a biolophysical or biomechanical attribute of an endogenous protein or peptide in the cellular or extracellular environment. A peptide that has a biological activity of an extracellular matrix protein or peptide is a peptide, motif, or residue that physically and/or biochemically acts like the extracellular matrix protein from which it is derived, thereby mimicking the protein or peptide. Extracellular matrix proteins with desirable biological activities include, for example: Fibronectin (RGD, GRGDSP, PHSRN), N-Cadherin (HAVDI), Heparin (Dextran sulfate), Collagen (IVH1, DGEA), Laminin (IKVAV, YIGSR).

Embodiment 17. The method of any one of Embodiments 1-16, wherein the integrated microenvironment recruits cells to and retains cells at the site of damaged tissue in the subject.

Embodiment 18. The method of Embodiment 17, wherein the cells are exogenous to the subject.

Embodiment 19. The method of Embodiment 17 or 18, wherein the cells are stem cells or progenitor cells. In a particular aspect, the cells are mesenchymal stem cells.

Embodiment 20. The method of any one of Embodiments 1-19, wherein the integrated microenvironment promotes fibrogenesis.

Embodiment 21. The method of Embodiment 20, wherein the administering the composition gives rise to an integrated living fibrous barrier at the site of damaged tissue in the subject.

Embodiment 22. The method of any one of Embodiments 1-21, wherein the administering the composition gives rise to a bioseal at the site of damaged tissue in the subject. As used herein, “bioseal” means an integrated barrier that is bound to the site of damaged tissue by cellular and fibrous contacts within the tissue. The barrier “seals” the site of damaged tissue and restores, for example, fluid pressurization within the tissue, including within the interstitial space within the tissue, and biomechanical properties of the tissue. The “bioseal” thereby helps to stop further tissue degeneration and promotes tissue healing.

Embodiment 23. The method of any one of Embodiments 1-22, wherein the composition is a hydrogel and the administering comprises locally applying the hydrogel at the site of damaged tissue. A person skilled in the art will appreciate certain pharmaceutically acceptable carriers, diluents, additives, excipients, binding agents, and the like that will be useful in formulating a hydrogel for use in the presently described methods.

Embodiment 24. The method of Embodiment 23, wherein the applying is performed arthroscopically.

Embodiment 25. The method of any one of Embodiments 1-22, wherein the composition is a liquid and the administering comprises injecting the liquid. In particular embodiments, injecting the liquid can comprise systemically injecting the liquid or locally injecting the liquid. Systemically injecting the liquid can comprise intravenously injecting. Locally injecting the liquid can comprise intramuscularly or subcutaneously injecting at or near the site of damaged tissue. In addition, the liquid can be injected directly into a joint of a subject for local administration of the liquid composition directly to an afflicted joint or a site of damaged tissue at a joint in the subject.

Embodiment 26. The method of any one of Embodiments 1-22, wherein the composition is an implantable solid and the administering comprises implanting the solid at the site of damaged tissue. A person skilled in the art will appreciate certain pharmaceutically acceptable carriers, diluents, additives, excipients, binding agents, and the like that will be useful in formulating an implantable solid for use in the presently described methods.

Embodiment 27. The method of any one of Embodiments 1-22, wherein the composition is an implantable paste and the administering comprises implanting the paste at the site of damaged tissue. A person skilled in the art will appreciate certain pharmaceutically acceptable carriers, diluents, additives, excipients, binding agents, and the like that will be useful in formulating an implantable paste for use in the presently described methods.

Embodiment 28. The method of Embodiment 9, wherein the cross-linking agent comprises ammonium persulfate (APS), dithiothreitol (DTT), or microbubbles comprising APS or DTT. In a particular embodiment, the microbubbles can be activated to release the APS or the DTT or another cross-linking agent upon application of or exposure to a cross-linking stimulus. The activation or release of the microbubbles can comprise bursting of the microbubbles to deliver the cross-linking agent to the surrounding tissue.

Embodiment 29. The method of Embodiment 28, further comprising the step of exposing the polymer to a cross-linking stimulus. The cross-linking stimulus can be any stimulus capable of inducing cross-linking of the polymers, including, for example, the modifications present on the polymers. The cross-linking stimulus can be, for example, light, or it can be chemical, i.e., a cross-linking agent.

Embodiment 30. The method of Embodiment 29, wherein the cross-linking stimulus is visible light, blue light, ultraviolet light, or ultrasound. The duration of the cross-linking stimulus can vary depending on the intensity of the stimulus, the cross-linking agent used, and/or the degree of cross-linking desired. In some embodiments, the polymer is exposed to the cross-linking stimulus for a few seconds, for example about 1 to about 30 seconds. In other embodiments, the duration of the cross-linking stimulus is on the order of a few minutes, for example, about 1 minute to about 5 minutes. In other embodiments, the duration of the cross-linking stimulus is about 5 minutes to about 15 minutes. In some embodiments, the duration can be about 15 minutes to about 30 minutes. In particular embodiments, the duration of the cross-linking stimulus is about 1 second, about 5 seconds, about 10 seconds, about 15 seconds, about 20 seconds, about 25 seconds, about 30 seconds, about 45 seconds, about 60 seconds, about 75 seconds, about 90 seconds, about 105 seconds, about 120 seconds, about 135 seconds, about 150 seconds, about 165 seconds, about 180 seconds, or more. In some embodiments, the duration of the cross-linking stimulus is about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, or about 15 minutes or more. In some embodiments, the duration of the cross-linking stimulus is about 20 minutes, about 25 minutes, about 30 minutes, about 35 minutes, about 40 minutes, about 45 minutes, about 50 minutes, about 55 minutes, or about 60 minutes. In some embodiments, the duration of the cross-linking stimulus is 1 hour or more. The duration of the cross-linking stimulus is any suitable amount of time appropriate for cross-linking the polymers, including for example the polymer modifications described herein, to the desired extent for the desired biomechanical constitution.

Embodiment 31. The method of any one of Embodiments 1-30, further comprising administering stem cells or progenitor cells to the subject. In a particular aspect, the cells are mesenchymal stem cells.

Embodiment 32. The method of any one of Embodiments 1-31, wherein the damaged tissue is cartilage, meniscus tissue, annulus fibrosis, ligament, or tendon.

Embodiment 33. The method of Embodiment 32, wherein the cartilage is debrided cartilage.

Embodiment 34. The method of Embodiment 32 or 33, wherein the subject has undergone meniscectomy.

Embodiment 35. The method of any one of Embodiments 32-34, wherein the cartilage exhibits tissue degeneration.

Embodiment 36. A composition for forming an integrated microenvironment at a site of damaged tissue in a subject, the composition comprising a polymer, wherein the composition further comprises one or more tunable characteristics such that the composition forms an integrated microenvironment having a desired biomechanical constitution at the site of damaged tissue in the subject. As provided above, the “integrated microenvironment” may be a region within a tissue that is adjacent to a tissue/environment interface that is characterized by interdigitation, interdispersion, or mixture between endogenous tissue components and biomaterial (i.e., the present compositions, or the polymer of the compositions) in accordance with the present disclosure. The resulting microenvironment can represent a new surface environment at the site of application of the composition to the tissue that can promote cell attachment, influence mechno-sensation, and/or direct cell response (e.g., promote matrix deposition) by providing biomechanical and/or biochemical support to cells at, on, or in the microenvironment. The one or more tunable characteristics of the composition can be tuned by a person skilled in the art to enable an integrated microenvironment that favors cell recruitment, cell growth, and/or cellular differentiation according to the present disclosure. As used herein, “tunable” means capable of being adjusted to have certain desired characteristics or properties.

Embodiment 37. The composition of Embodiment 36, wherein the one or more tunable characteristics comprise: a tunable polymer composition; a tunable polymer concentration; a tunable polymer modification; a tunable polymer conjugation; a tunable polymer cross-linking; or a tunable polymer stiffness. As described above in Embodiment 2, the tunable characteristics can be selected based, for example, on a particular target tissue, on the biomechanical properties of the target tissue, on the biochemical properties of the target tissue, or on the desired characteristics or properties of the integrated microenvironment to be formed at the site of damaged tissue.

With respect to Embodiment 36, the polymer may comprise a biopolymer, a polysaccharide, a synthetic polymer, or any combination thereof. Biopolymers may comprise collagen, gelatin, albumin, cellulose, or any combination thereof. Polysaccharides may comprises hyaluronic acid, dextran, chondroitin sulfate, heparin, dermatan, or any combination thereof. Synthetic polymers may comprise polyethylene glycol (PEG), poly-L-lactic acid (PLLA), polylacticglycolic acid (PLGA), polyglycolic acid (PGA), polycaprolactone (PCL), polyethylene oxide (PEO), polyvinyl alcohol (PVA), polyethylene, polypropylene, a polyacrylate, a polycarbonate, or any combination thereof

In the present compositions, the tunable polymer modification may comprise an aldehyde modification, a methacrylate modification, a norbornene modification, or a tyramine modification. Methods useful for producing polymers having the modifications described herein include, but are not limited to, sodium periodate oxidation, methacrylic anhydride methacrylation, peptide conjugation via Michael addition, norbornene modification (BOP coupling), and carbodiimide coupling (e.g tyramine).

In some embodiments of the present compositions, the tunable polymer cross-linking may comprise a cross-linkable polymer, a cross-linkable polymer modification, or a cross-linking agent.

Regarding the tunable polymer composition, the polymer, the polymer concentration, and the polymer molecular weight can be tuned to control cellular stiffness of the cellular microenvironment and cellular response. For example, the concentration of polymer in the composition can be tuned, allowing for the amount of polymer added to and the ultimate density and mechanics of the microenvironment to be controlled. Additionally or alternatively, the molecular weight can also be tuned, altering diffusion of the polymer into the damaged tissue (e.g., the cartilage tissue), and ultimately the foundation of the integrated microenvironment.

Regarding the tunable polymer modification, the polymers can be modified with a peptide. The peptide concentration and type (RGD, HAVDI) can be tuned to control cellular response. For example, adding RGD groups to the polymer promotes cellular attachment and spreading. Other peptides containing proteins or growth factors can tune stem cells to differentiate toward a desired phenotype (e.g. chondrogenesis, fibrogenesis, osteogenesis, etc.). Any peptide with a thiol can be added to the polymers. Peptides that have a biological activity of an extracellular matrix protein are particularly desirable. An activity of a protein can be a biolophysical or biomechanical attribute of an endogenous protein or peptide in the cellular or extracellular environment. A peptide that has a biological activity of an extracellular matrix peptide is a peptide, motif, or residue that physically and/or biochemically acts like the extracellular matrix protein from which it is derived, thereby mimicking the protein. Extracellular matrix proteins with desirable biological activities include, for example: Fibronectin (RGD, GRGDSP, PHSRN), N-Cadherin (HAVDI), Heparin (Dextran sulfate), Collagen (IVH1, DGEA), Laminin (IKVAV, YIGSR).

Regarding the tunable stiffness: the stiffness of the cellular microenvironment can control the response of adhered stem cells through mechano-sensation. In addition, mechanics of degenerated tissue can be at least partially restored or fully restored by tuning the stiffness of the cellular microenvironment. Stiffness of the microenvironment is typically measured via atomic force microscopy (AFM), providing mechanics at the cell-scale level. A chemical modification can be selected based on desired properties of the cellular microenvironment. For example, methacrylation and/or norbomene modification can be used to tune the amount of cross-linking. In addition, the duration of cross-linking time can be tuned to achieve a desired stiffness of the cellular microenvironment. Increased methacrylation of the polymers will cross-link to a higher stiffness than polymers having relatively less methacrylation. Based on degree of methacrylation and UV cross-linking time, the stiffness of the biomaterial itself can be varied from <1 to >50 kPa. When applied to either non-degraded or degraded tissue, the cross-linked biomaterial can provide 50-100% improvement in mechanics. The polymers can be modified to have aldehyde substitutions. The adjacent hydroxyl groups on the polymers can be modified to present aldehydes, which can bind to exposed amines in damaged tissues. The degree of aldehyde substitution (and/or tyramine in an alternative embodiment) can be varied to modify binding to the exposed damaged tissue. Tyramine substitution can also be conjugated to the backbone of the polymers, and allow for a range of attachment to damaged cartilage, for example, thereby cross-linking of the integrated microenvironment, and conjugation of peptides.

In addition to the protein biomaterial, the present compositions can comprise a carrier. The carrier may be, for example, water, a buffer solution (e.g., PBS), culture medium, an alcohol (e.g., ethanol), an acid, an organic solvent, or any combination thereof

Embodiment 38. The composition of Embodiment 37, wherein the tunable polymer modification binds to the site of damaged tissue.

Embodiment 39. The composition of any one of Embodiments 36-38, wherein the tunable polymer conjugation is a therapeutic conjugate.

Embodiment 40. The composition of Embodiment 39, wherein the therapeutic conjugate comprises a drug. The drug can be, for example, an anti-inflammatory, an anti-histamine, an anti-biotic, transforming growth factor (TGF)-β, (SDF), bone morphogenic protein (BMP), or a pain killer. Appropriate drugs will be readily appreciated by persons skilled in the art.

Embodiment 41. The composition of Embodiment 40, wherein the therapeutic conjugate comprises a peptide having a thiol group.

Embodiment 42. The composition of Embodiment 41, wherein the peptide comprises an RGD cell adhesion domain.

Embodiment 43. The composition of Embodiment 42, wherein the peptide is a growth factor.

Embodiment 44. The composition of Embodiment 43, wherein the peptide comprises an extracellular matrix peptide or a derivative of an extracellular matrix peptide.

Embodiment 45. The composition of Embodiment 42, wherein the extracellular matrix peptide comprises one or more peptide activities of fibronectin, N-cadherin, heparin, collagen, or laminin, or combinations thereof. As described above in Embodiment 16, a peptide activity can be a biolophysical or biomechanical attribute of an endogenous protein or peptide in the cellular or extracellular environment. A peptide that has a biological activity of an extracellular matrix protein or peptide is a peptide, motif, or residue that physically and/or biochemically acts like the extracellular matrix protein from which it is derived, thereby mimicking the protein or peptide. Extracellular matrix proteins with desirable biological activities include, for example: Fibronectin (RGD, GRGDSP, PHSRN), N-Cadherin (HAVDI), Heparin (Dextran sulfate), Collagen (IVH1, DGEA), Laminin (IKVAV, YIGSR).

1. A method of repairing damaged tissue in a subject, comprising: administering to the subject a composition according to claim 36.

2. (canceled)

3. The method of claim 1, further comprising ascertaining one or more aspects of the desired biomechanical constitution of the integrated microenvironment, and tuning one or more of the tunable characteristics of the composition comprising the polymer to give rise to the desired biomechanical constitution.

4.-35. (canceled)

36. A composition for forming an integrated microenvironment at a site of damaged tissue in a subject, the composition comprising a polymer, wherein the composition further comprises one or more tunable characteristics such that the composition forms an integrated microenvironment having a desired biomechanical constitution at the site of damaged tissue in the subject.

37. The composition of claim 36, wherein the one or more tunable characteristics comprise:
a tunable polymer composition;
a tunable polymer concentration;
a tunable polymer modification;
a tunable polymer conjugation;
a tunable polymer cross-linking; or
a tunable polymer stiffness.

38. The composition of claim 37, wherein the tunable polymer modification binds to the site of damaged tissue.

39. The composition of claim 36, wherein the tunable polymer conjugation is a therapeutic conjugate.

40. The composition of claim 39, wherein the therapeutic conjugate comprises a drug.

41. The composition of claim 40, wherein the therapeutic conjugate comprises a peptide having a thiol group.

42. The composition of claim 41, wherein the peptide comprises an RGD cell adhesion domain.

43. The composition of claim 42, wherein the peptide is a growth factor.

44. The composition of claim 43, wherein the peptide comprises an extracellular matrix peptide or a derivative of an extracellular matrix peptide.

45. The composition of claim 42, wherein the extracellular matrix peptide comprises one or more peptide activities of fibronectin, N-cadherin, heparin, collagen, or laminin, or combinations thereof.

46. The composition of claim 36, wherein the polymer comprises hyaluronic acid, dextran, chondroitin sulfate, heparin, dermatan, or any combination thereof

47. The composition of claim 36, wherein the polymer comprises hyaluronic acid.

48. The composition of claim 36, wherein the polymer is modified with one or more of aldehyde, tyramine, or methacrylate groups.

49. The composition of claim 48, wherein the polymer is modified with aldehyde and methacrylate groups.

50. The composition of claim 36, wherein the polymer is present in the composition at a concentration of about 1 to about 20% w/v.

51. The composition of claim 36, wherein the polymer is present in the composition at a concentration of about 1 to about 15% w/v.

52. The composition of claim 36, wherein the polymer is conjugated to a chemoattractant molecule or peptide.

53. The composition of claim 36, wherein the polymer is conjugated to RGD.

54.-60. (canceled)
